(19) United States
(12) Patent Application Publication
(10) Pub. No.: US 2021/0196925 A1
(43) Pub. Date: Jul. 1, 2021

(54) CATHETER ASSEMBLY

(71) Applicant: TERUMO KABUSHIKI KAISHA, Tokyo (JP)

(72) Inventor: Masahiro ISHIDA, Hadano-shi (JP)

(73) Assignee: TERUMO KABUSHIKI KAISHA, Tokyo (JP)

(21) Appl. No.: 17/200,025

(22) Filed: Mar. 12, 2021

Related U.S. Application Data
(63) Continuation of application No. PCT/JP2019/032440, filed on Aug. 20, 2019.

(30) Foreign Application Priority Data
Sep. 19, 2018 (JP) 2018-174970

(51) Int. Cl. A61M 25/00; A61M 25/06

(52) U.S. Cl. CPC A61M 25/0054; A61M 25/0693; A61M 25/0606; A61M 25/0074

(57) ABSTRACT

A catheter assembly includes a catheter and an inner needle inserted through the catheter. The catheter includes a plurality of sites that are distinguishable from each other along the axial direction. The plurality of sites have different hardnesses between adjacent sites.

### CROSS-REFERENCE TO RELATED APPLICATIONS

This application is a bypass continuation of PCT Application No. PCT/JP2019/032440, filed on Aug. 20, 2019, which claims priority to Japanese Application No. 2018-174970, filed on Sep. 19, 2018. The contents of these applications are hereby incorporated by reference in their entireties.

### BACKGROUND

The present disclosure relates to a catheter assembly configured to puncture and indwell in a blood vessel when performing an infusion or the like to a patient, for example.

Conventionally, a catheter assembly used when performing an infusion or the like to a patient has been known. This kind of the catheter assembly includes a hollow catheter, a catheter hub fixed to a proximal end of the catheter, a hollow inner needle that is inserted into the catheter and has a sharp needle tip at a distal end, and a needle hub fixed to a proximal end of the inner needle (for example, see JP 2008-43445 A). When using a catheter assembly, skin and a blood vessel of a living body are punctured with each distal end of the inner needle and the catheter, and the catheter is then advanced with respect to the inner needle so that the catheter is inserted into the blood vessel by a predetermined length.

### SUMMARY

Certain embodiments of the present invention have been developed in connection with the above-described related art, and one object thereof is to provide a catheter assembly capable of easily imparting desired characteristics to a catheter according to a therapeutic purpose and an application.

According to one embodiment, a catheter assembly includes: a catheter; and an inner needle inserted through the catheter. The catheter has a plurality of sites that are distinguishable from each other along an axial direction. The plurality of sites have different hardnesses between adjacent sites.

According to certain embodiments of the catheter assembly, the hardness differs between adjacent sites of the plurality of sites provided on the catheter, and thus, it is possible to easily impart the desired characteristics to the catheter according to the therapeutic purpose and the application.

### BRIEF DESCRIPTION OF DRAWINGS

FIG. 1 is a perspective view of a catheter assembly according to a first embodiment of the present invention;

FIG. 2 is a cross-sectional view of a distal portion of the catheter assembly;

FIG. 3 is an explanatory view of a function of the catheter assembly;

FIG. 4 is a cross-sectional view of a distal portion of a catheter assembly according to a second embodiment of the present invention;

FIG. 5 is a cross-sectional view of a distal portion of a catheter assembly according to a third embodiment of the present invention;

FIG. 6 is a cross-sectional view of a distal portion of a catheter assembly according to a fourth embodiment of the present invention;

FIG. 7 is a cross-sectional view of a distal portion of a catheter assembly according to a fifth embodiment of the present invention;

FIG. 8 is a cross-sectional view of a distal portion of a catheter assembly according to a sixth embodiment of the present invention; and

FIG. 9 is a cross-sectional view of a distal portion of a catheter assembly according to a seventh embodiment of the present invention.

### DETAILED DESCRIPTION

Hereinafter, preferred embodiments of a catheter assembly according to the present invention will be described with reference to the accompanying drawings.

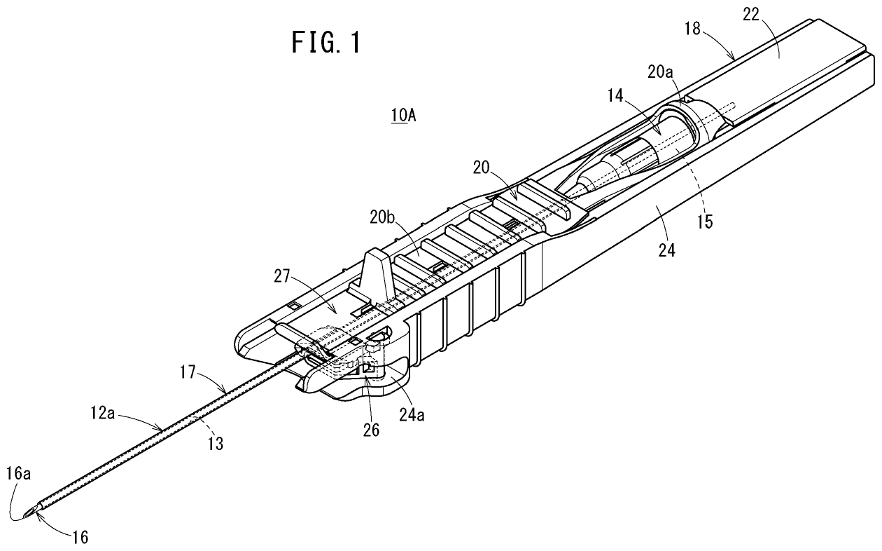

A catheter assembly 10A whose initial state is illustrated in FIG. 1 is applied when performing an infusion, a blood transfusion, and the like to a patient (living body), and forms an introduction portion for a medicinal liquid or the like by puncturing the patient's body to indwell. The catheter assembly 10A may be configured as a catheter (for example, a central venous catheter, a PICC, a mid-line catheter, and the like) having a longer length than a peripheral venous catheter. Alternatively, the catheter assembly 10A may be configured as a peripheral venous catheter. In addition, the catheter assembly 10A is not limited to the venous catheter, and may be configured as an arterial catheter such as a peripheral arterial catheter.

As illustrated in FIG. 1, the catheter assembly 10A includes a catheter 12a, a catheter hub 14 fixedly holding the catheter 12a, a hollow inner needle 16 removably inserted into the catheter 12a, a needle hub 18 fixedly holding the inner needle 16, and a catheter operation member 20 mounted to the catheter hub 14. The inner needle 16 may be a solid needle.

The catheter assembly 10A forms a multi-tube structure (multi-tube portion) in which the catheter 12a and the inner needle 16 are sequentially stacked in an initial state before use.

The catheter 12a is flexible and has a lumen 13 that extends therethrough. The lumen 13 is formed to have a diameter capable of accommodating the inner needle 16 and capable of causing a medicinal liquid, blood, or the like to flow. A distal end of the catheter 12a is reduced in diameter in order to decrease a puncture resistance, and an inner surface of the catheter 12a is in close contact with an outer surface of the inner needle 16 at such a reduced diameter portion in the initial state of the catheter assembly 10A. A length of the catheter 12a is not particularly limited but can be appropriately designed according to use and various conditions, and is set to, for example, about 14 to 500 mm, about 30 to 400 mm, or about 76 to 200 mm.

A proximal portion of the catheter 12a is fixed to a distal portion inside the catheter hub 14. The catheter 12a and the catheter hub 14 form a catheter member 17.

The catheter hub 14 is exposed on the patient's skin in a state in which the catheter 12a has been inserted into a blood vessel, and indwelled together with the catheter 12a by being affixed with a tape or the like. The catheter hub 14 is formed in a tubular shape that is tapered in a distal direction.

A constituent material of the catheter hub 14 is not particularly limited, but a thermoplastic resin, such as polypropylene, polycarbonate, polyamide, polysulfone, polyarylate, a methacrylate-butylene-styrene copolymer, and polyurethane may be preferably used.

A hollow portion 15 that communicates with the lumen 13 of the catheter 12a and through which an infusion solution can flow is provided inside the catheter hub 14. A hemostatic valve, a plug, or the like (not illustrated) may be accommodated inside the hollow portion 15 in order to prevent back-flow of blood at the time of puncture with the inner needle 16 and to allow infusion along with insertion of a connector of an infusion tube.

The inner needle 16 is configured as a hollow tube having rigidity that enables puncture of skin of a living body, and is arranged to penetrate through the lumen 13 of the catheter 12a and the hollow portion 15 of the catheter hub 14. The inner needle 16 is formed to have a total length longer than that of the catheter 12a, and a sharp needle tip 16a is provided at a distal end thereof. A lumen penetrating in an axial direction of the inner needle 16 is provided inside the inner needle 16, and this lumen communicates with a distal opening of the inner needle 16.

Examples of a constituent material of the inner needle 16 include a metal material such as stainless steel, aluminum or an aluminum alloy, and titanium or a titanium alloy, a hard resin, ceramics, and the like.

The needle hub 18 has a needle holding member 22 fixed to a proximal portion of the inner needle 16, and a housing 24 to which the needle holding member 22 is fixed and that extends along the inner needle 16 and the catheter 12a. In the initial state of the catheter assembly 10A, the housing 24a houses part of the multi-tube structure, the catheter hub 14, and the catheter operation member 20. Resin materials forming the needle holding member 22 and the housing 24 are not particularly limited. However, for example, the materials exemplified for the catheter hub 14 can be appropriately selected. Incidentally, the needle holding member 22 and the housing 24 may be monolithically formed.

When the needle hub 18 is moved to a proximal direction with respect to the catheter 12a, the inner needle 16 is also moved in the proximal direction with respect to the catheter 12a along with the movement of the needle hub 18 because the needle hub 18 holds the inner needle 16 at the needle holding member 22.

The catheter operation member 20 is attached to the catheter hub 14. Thus, when the catheter operation member 20 is advanced relative to the needle hub 18, the catheter member 17 is advanced relative to the inner needle 16. The catheter operation member 20 has a hub mounting portion 20a detachably mounted on the catheter hub 14, and an operation plate portion 20b extending from the hub mounting portion 20a along the catheter 12a in the distal direction. Incidentally, the catheter operation member 20 is not necessarily provided in the catheter assembly 10A.

The catheter assembly 10A is provided with a support member 26 on the distal side of the housing 24 in order to support a lower side of the catheter 12a held by the catheter operation member 20. The support member 26 is rotatably attached to an arrangement concave portion 24a provided at a distal portion of the housing 24. A distal portion of the catheter operation member 20 and the support member 26 constitute a deflection suppressing mechanism 27.

When the skin is punctured with the inner needle 16 and the catheter 12a, the distal portion of the catheter operation member 20 supports the catheter 12a from above and the support member 26 supports the catheter 12a from below, and thus, deflection of the catheter 12a and the inner needle 16 is suppressed. When the catheter operation member 20 is removed from the housing 24, the support member 26 is rotated toward an outer side of the housing 24 by being pushed by the hub mounting portion 20a, and thus, the catheter hub 14 can be withdrawn from the housing 24 in the distal direction. Incidentally, the support member 26 is not necessarily provided.

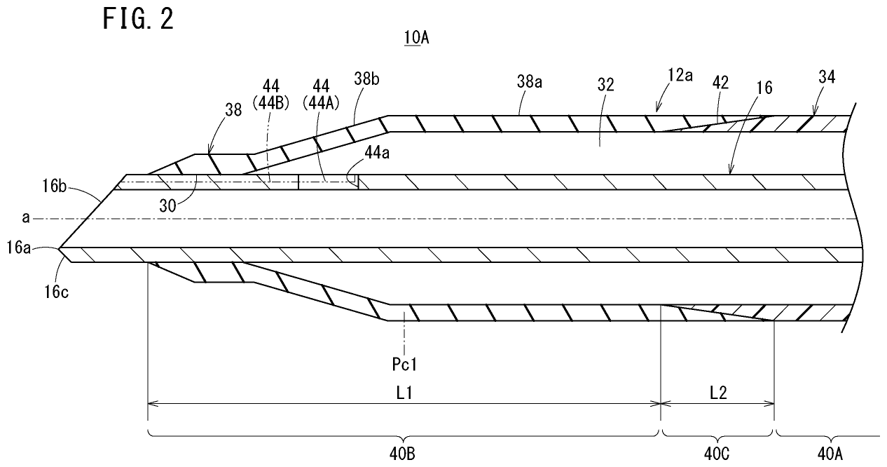

As illustrated to FIG. 2, the catheter 12a has a close contact portion 30, which is in close contact with an outer circumferential surface of the inner needle 16 over the entire circumference, in at least a part of an inner circumferential surface. The close contact portion 30 is provided on the inner circumferential surface of a distal portion of the catheter 12a. A flow path for flashback confirmation (hereinafter, referred to as “flashback flow path 32”) is formed between the catheter 12a and the inner needle 16 on the proximal side of the close contact portion 30. The flashback flow path 32 extends up to a proximal opening of the catheter 12a.

The catheter 12a has a plurality of sites that can be distinguished from each other along a direction of an axis a of the catheter (hereinafter, referred to as “axial direction”). The plurality of sites have different hardness (elastic moduli) between adjacent sites. In a first embodiment, the catheter 12a has, as the plurality of sites, a catheter body 34 constituting the main part of the catheter 12a and a flexible portion 38 provided at a distal portion of the catheter body 34. Thus, the catheter 12a becomes more flexible toward the distal-most portion on the distal side. The flexible portion 38 is exposed from the housing 24 (FIG. 1).

The catheter body 34 accounts for most of the whole length of the catheter 12a. Thus, the distal-most portion of the catheter body 34 is positioned near the distal-most end of the catheter 12a. The catheter body 34 and the flexible portion 38 are made of a resin material having flexibility.

The flashback flow path 32 is formed between an inner circumferential surface of the catheter body 34 and an outer circumferential surface of the inner needle 16. Among the catheter body 34 and the flexible portion 38, at least the catheter body 34 has transparency such that a flashback can be confirmed.

The catheter 12a is supported by the support member 26 (FIG. 1) at a spot of the catheter body 34 (the catheter body 34 is supported by the support member 26). As a result, it is possible to reliably support the catheter 12a and to reduce a sliding resistance at the time of advancing the catheter 12a. Moreover, the portion supported by the support member 26 (FIG. 1) is located on the proximal side of an interface 42 between the catheter body 34 and the flexible portion 38, and thus, it is possible to prevent peeling of the interface 42 caused by sliding of the catheter 12a with respect to the support member 26.

It is preferably that the catheter body 34 be less likely to swell as compared with the flexible portion 38. As a result, it is possible to set an axial distance between a distal-most position of the inner needle 16 and a distal-most position of the catheter 12a to a desired size and to reduce a variation for each product during steam sterilization (autoclave sterilization) or ethylene oxide gas sterilization in a process of manufacturing the catheter assembly 10A.

Examples of a constituent material of the catheter body 34 include a fluorine-based resin such as polytetrafluoroethylene (PTFE), an ethylene-tetrafluoroethylene copolymer (ETFE), and a perfluoroalkoxy fluorine resin (PFA), an olefin-based resin such as polyethylene and polypropylene or a mixture thereof, polyurethane, polyester, polyamide, a polyether nylon resin, a mixture of the olefin-based resin and an ethylene-vinyl acetate copolymer, and the like. A durometer hardness of the catheter body 34 is, for example, less than D70.

The flexible portion 38 includes the distal-most portion of the catheter 12a. The flexible portion 38 is more flexible than the catheter body 34. That is, an elastic modulus k1 of the catheter body 34 and an elastic modulus k2 of the flexible portion 38 have a relationship of k1>k2.

The flexible portion 38 has: a straight portion 38a that has a constant outer diameter along the axial direction; and a tapered portion 38b that extends from the straight portion 38a in the distal direction and has an outer diameter that decreases in the distal direction. An inner circumferential surface of the flexible portion 38 is in close contact with (fitted to) an outer circumferential surface of the inner needle 16 in a liquid-tight manner over the entire outer circumference.

It is preferable that at least the flexible portion 38 between the catheter body 34 and the flexible portion 38 have an X-ray contrast property. As a result, for example, when the catheter 12a is broken in a blood vessel, it is possible to easily confirm a location of the catheter 12a that has been broken and left in the blood vessel by X-ray. A contrast layer in the case where the flexible portion 38 has the contrast property may be provided, for example, in any form of a stripe shape, an intermediate layer in the radial direction, or the whole layer.

Examples of a constituent material of the flexible portion 38 include various rubber materials such as natural rubber, butyl rubber, isoprene rubber, butadiene rubber, styrene-butadiene rubber, silicone rubber, various thermoplastic elastomers such as polyurethanes, polyesters, polyamides, olefins, and styrenes or a mixture thereof, and the like.

In the catheter 12a, a single catheter body region 40A where only the catheter body 34, among the catheter body 34 and the flexible portion 38, exists, a single flexible portion region 40B where only the flexible portion 38, among the catheter body 34 and the flexible portion 38, exists, and a mixed region 40C where the catheter body 34 and the flexible portion 38 exist, are arranged in the axial direction. In the catheter 12a illustrated in FIG. 2, the interface 42 between the catheter body 34 and the flexible portion 38 is formed in a tapered shape that is inclined at a substantially constant angle with respect to an axis a of the catheter 12a.

The single catheter body region 40A is a portion of the catheter body 34 present on the proximal side of a proximal-most portion of the flexible portion 38.

The single flexible portion region 40B is a portion of the flexible portion 38 present on the distal side of the distal-most portion of the catheter body 34. An axial length L1 of the single flexible portion region 40B is set to, for example, 0.3 to 5.0 mm, preferably 0.4 to 2.0 mm, and more preferably 0.5 to 0.9 mm. The hardness of the flexible portion (the single flexible portion region 40B) is, for example, A80 to D67 and preferably D53 to D64 at 23° C. The flexible portion 38 in the illustrated example is joined to the catheter body 34. Because the axial length and the hardness of the single flexible portion region 40B are set within the above ranges, it is possible to prevent the distal end (the flexible portion 38) of the catheter 12a from being curled at the time of puncture. In addition, it is possible to preferably suppress catching by a blood vessel back wall at the time of inserting the catheter 12a. Further, it is possible to suppress crushing of the distal end of the catheter 12a at the time of suctioning blood.

The mixed region 40C is a portion in which the catheter body 34 and the flexible portion 38 are stacked in the radial direction. An axial length L2 of the mixed region 40C is set to, for example, 1 to 5 mm, and preferably 2 to 3 mm.

In the catheter 12a illustrated in FIG. 2, the interface 42 between the catheter body 34 and the flexible portion 38 is inclined in the distal direction so as to approach the axis a (center) of the catheter 12a. Thus, the flexible portion 38 is present on the outer side of the catheter body 34 in the mixed region 40C.

Instead of the above configuration having the interface 42, the catheter 12a may be formed so as to become soft in the distal direction by changing each compounding amount of materials different in hardness in the axial direction. In this case, extrusion molding may be performed while changing each extrusion speed of different materials. Alternatively, a content of a plasticizer at the distal portion of the catheter 12a may be increased. In this case, the plasticizer may be applied to the distal portion of the catheter 12a.

In the inner needle 16, a backcut portion 16c, which is continuous with the needle tip 16a and is inclined in a reverse direction to the blade face 16b with respect to the axis of the inner needle 16, is provided on the opposite side to a blade face 16b.

The inner needle 16 is provided with an introduction path 44 that communicates with the flashback flow path 32 to introduce blood into the flashback flow path 32. The introduction path 44 illustrated by a solid line in FIG. 2 is a side hole 44A that penetrates through a wall portion of the inner needle 16 in the radial direction. As illustrated by an imaginary line in FIG. 2, the introduction path 44 may be a groove portion 44B extending in the axial direction on the outer circumferential surface of the inner needle 16.

In the initial state of the catheter assembly 10A illustrated in FIG. 2, at least a proximal end 44a of the introduction path 44 is provided on the distal side of the distal-most portion of the single catheter body region 40A. The entire side hole 44A illustrated in FIG. 2 is provided on the distal side of the distal-most portion of the single catheter body region 40A. More specifically, the proximal end 44a of the introduction path 44 is provided on the distal side of an axial center position Pc1 of the single flexible portion region 40B. The proximal end 44a of the introduction path 44 is provided on the distal side of a proximal end of the tapered portion 38b of the flexible portion 38. The side hole 44A is provided at a position where the entire side hole 44A opposes an inner circumferential surface of the tapered portion 38b of the flexible portion 38. In another aspect, the proximal end 44a (or the entire side hole 44A) of the introduction path 44 may be provided at a position opposing an inner circumferential surface of the straight portion 38a of the flexible portion 38.

Regarding a position of the introduction path 44 in relation to the close contact portion 30, at least the proximal end 44a of the introduction path 44 (the side hole 44A or the groove portion 44B) is provided on the proximal side of the close contact portion 30 in the initial state of the catheter assembly 10A illustrated in FIG. 2. The entire side hole 44A is provided on the proximal side of the close contact portion 30.

Next, functions of the catheter assembly 10A configured as described above will be described.

In use of the catheter assembly 10A illustrated in FIG. 1, a puncturing operation to puncture the patient's skin with the catheter assembly 10A is performed. In the puncturing operation, a user (a doctor, a nurse, or the like) presses the distal portion of the catheter assembly 10A against the patient while gripping the housing 24, thereby puncturing the skin toward a puncture target blood vessel. Accordingly, the skin is punctured with each distal portion of the inner needle 16 and the catheter 12a.

Next, the user operates the catheter operation member 20 in the distal direction to cause the catheter member 17 (the catheter 12a and the catheter hub 14) to advance while fixing the position of the needle hub 18 (the housing 24). Accordingly, the catheter 12a is inserted to the target position in the blood vessel.

Next, the user pulls the housing 24 in the proximal direction while holding the positions of the catheter operation member 20 and the catheter member 17. Accordingly, the catheter member 17 and the catheter operation member 20 completely come out of the housing 24, and the inner needle 16 is removed from the catheter 12a in the proximal direction.

Next, the catheter operation member 20 is detached from the catheter hub 14. Accordingly, the catheter member 17 is indwelled in the patient. Incidentally, the catheter operation member 20 may be kept attached to the catheter hub 14 depending on a preference of the user.

Next, the connector of the infusion tube (not illustrated) is connected to the proximal side (the proximal portion of the catheter hub 14) of the catheter member 17 from which the inner needle 16 has been removed, and the infusion solution (medicinal liquid) is administered from the infusion tube to the patient.

In this case, the catheter assembly 10A according to the present embodiment has the following effects.

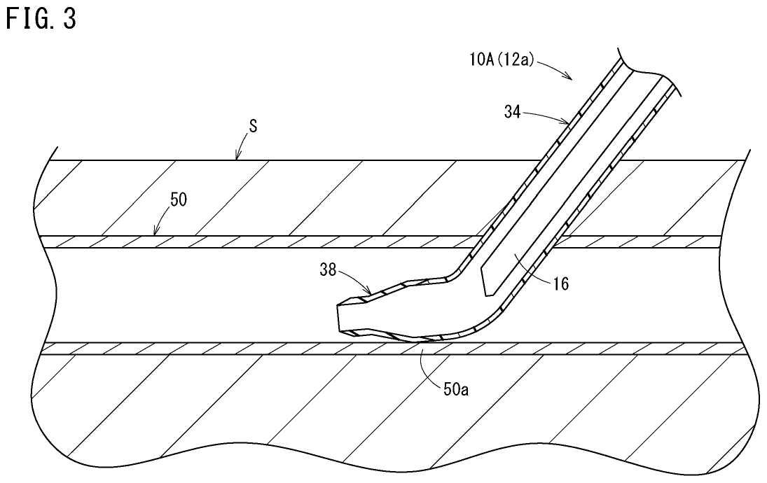

According to the catheter assembly 10A, the flexible portion 38, which is more flexible than the catheter body 34, is provided at the distal portion of the catheter body 34 as illustrated in FIG. 2. Thus, it is possible to prevent the distal end of the catheter 12a from being caught by a blood vessel back wall 50a, which is a blood vessel wall of the blood vessel 50 on the opposite side of a puncture spot at the time of advancing the catheter 12a to insert the catheter 12a into the blood vessel 50 after puncturing a skin S with the distal portion of the catheter assembly 10A even when a puncture angle, which is an angle between a central axis of the blood vessel 50 to be punctured and a central axis of the inner needle 16 with which the puncture is performed, is large as illustrated in FIG. 3.

That is, the flexible portion 38 is brought into contact with the blood vessel back wall 50a and is pressed by the blood vessel back wall 50a to be easily deformed at the time of advancing the catheter 12a as illustrated in FIG. 3, and thus, it is possible to prevent the distal end of catheter 12a from being caught by the blood vessel back wall 50a. As a result, it is possible to prevent the catheter 12a from being hardly inserted into the blood vessel 50 or to prevent the blood vessel back wall 50a from being damaged by the distal end of the catheter 12a.

As illustrated in FIG. 2, the flashback flow path 32 is formed between the catheter 12a and the inner needle 16, and the inner needle 16 is provided with the introduction path 44 that communicates with the flashback flow path 32 to introduce blood into the flashback flow path 32. The proximal end 44a of the introduction path 44 is provided on the distal side of the axial center position Pc1 of the portion of the flexible portion 38 present on the distal side of the distal-most portion of the catheter body 34. Thus, the flashback of blood can be confirmed earlier.

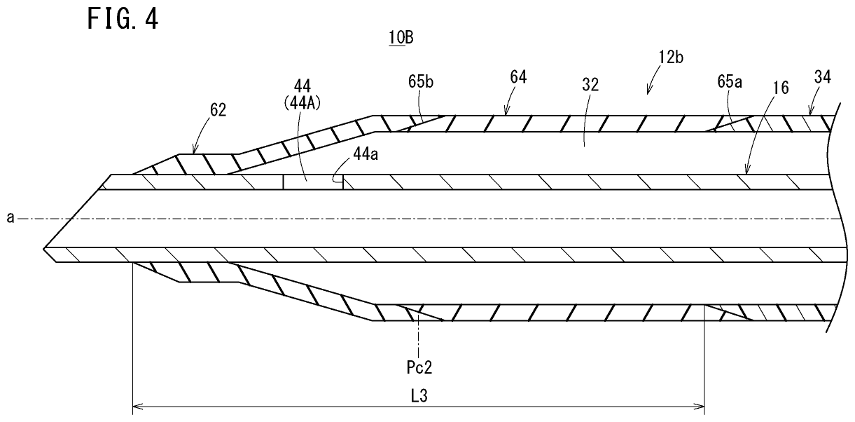

In a catheter assembly 10B according to a second embodiment illustrated in FIG. 4, a catheter 12b includes a plurality of sites that can be distinguished from each other along the axial direction. The plurality of sites have different hardness (elastic moduli) between adjacent sites. In the second embodiment, the catheter 12b includes, as the plurality of sites, the catheter body 34, a catheter distal portion 62 including a distal-most portion of catheter 12b, and a catheter intermediate portion 64 provided between the catheter body 34 and the catheter distal portion 62.

The catheter distal portion 62 is configured in the same manner as the flexible portion 38 in the first embodiment. Therefore, the catheter distal portion 62 is more flexible than the catheter body 34. The catheter intermediate portion 64 has a straight shape whose outer diameter at an outer circumferential surface of the catheter 12b is constant along the axial direction. In another aspect, the catheter intermediate portion 64 may have a tapered portion whose outer diameter at the outer circumferential surface of the catheter 12b decreases in the distal direction.

In one aspect, the catheter intermediate portion 64 has a hardness different from that of the catheter body 34 and the catheter distal portion 62. The catheter intermediate portion 64 may be configured to be more flexible than the catheter body 34 and may be configured to be stiffer than the catheter body 34. The catheter intermediate portion 64 may be configured to be more flexible than the catheter distal portion 62 or may be stiffer than the catheter distal portion 62. The catheter intermediate portion 64 may be configured to have an intermediate hardness between the catheter body 34 and the catheter distal portion 62, may be configured to be stiffer than both the catheter body 34 and the catheter distal portion 62, or may be configured to be more flexible than both the catheter body 34 and the catheter distal portion 62.

In another aspect, the catheter distal portion 62 may be configured to have the same hardness as the catheter body 34, or may be configured to be stiffer than the catheter body 34.

An interface (first interface 65a) between the catheter body 34 and the catheter intermediate portion 64 is formed in a tapered shape that is inclined so as to approach the axis a of the catheter 12b in the distal direction. An interface (second interface 65b) between the catheter distal portion 62 and the catheter intermediate portion 64 is formed in a tapered shape that is inclined so as to approach the axis a of the catheter 12b in the distal direction. An inclination angle of the first interface 65a with respect to the axis a of the catheter 12b and an inclination angle of the second interface 65b with respect to the axis a of the catheter 12b may be the same or different from each other.

In the initial state of the catheter assembly 10B, at least the proximal end 44a of the introduction path 44 is provided on the distal side of the distal-most portion of the catheter body 34. The entire side hole 44A is provided on the distal side of the distal-most portion of the catheter body 34. The proximal end 44a of the introduction path 44 is provided on the distal side of an axial center position Pc2 of a portion (region having a length L3 in FIG. 4), present on the distal side of the distal-most portion of the catheter body 34, in the combined region of the catheter distal portion 62 and the catheter intermediate portion 64. The entire side hole 44A is provided on the distal side of the distal-most portion of the catheter intermediate portion 64

In this manner, the plurality of sites provided on the catheter 12b have different hardnesses between adjacent sites in the catheter assembly 10B. Thus, the catheter 12b can be easily imparted with desired characteristics according to a therapeutic purpose and an application, which is similar to the first embodiment.

The catheter 12b includes, as the plurality of sites provided along the axial direction, the catheter body 34, the catheter distal portion 62, including the distal-most portion of catheter 12b, and the catheter intermediate portion 64 provided between the catheter body 34 and the catheter distal portion 62. With this configuration, the catheter body 34 and the catheter intermediate portion 64 have different hardness, and the catheter distal portion 62 and the catheter intermediate portion 64 have different hardness, and thus, it is easy to impart more complicated characteristics to the catheter 12b.

The proximal end 44a of the introduction path 44 is provided on the distal side of the axial center position Pc2 of the portion, present on the distal side of the distal-most portion of the catheter body 34, in the combined region of the catheter distal portion 62 and the catheter intermediate portion 64. With this configuration, the flashback of blood can be confirmed earlier.

Incidentally, the same or similar functions and effects as those of the first embodiment can be obtained in the second embodiment for common parts with the first embodiment

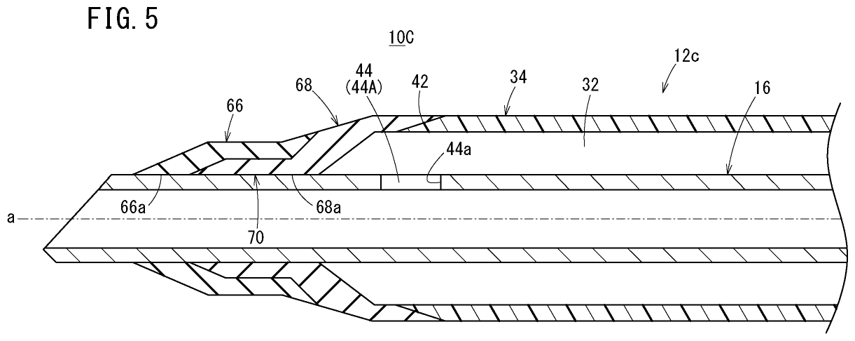

In a catheter assembly 10C according to a third embodiment illustrated in FIG. 5, a catheter 12c includes a plurality of sites that can be distinguished from each other along the axial direction. The plurality of sites have different hardness (elastic moduli) between adjacent sites. In the third embodiment, the catheter 12c includes, as the plurality of sites, the catheter body 34, a catheter distal portion 66 including a distal-most portion of catheter 12c, and a catheter intermediate portion 68 provided between the catheter body 34 and the catheter distal portion 66. The magnitude relationship of the hardness among the catheter body 34, the catheter distal portion 66, and the catheter intermediate portion 68 in the catheter 12c may be set in the same manner as the magnitude relationship of the hardness among the catheter body 34, the catheter distal portion 62, and the catheter intermediate portion 64 in the catheter 12b (FIG. 4) of the second embodiment.

The catheter 12c has a close contact portion 70, which is in close contact with an outer circumferential surface of the inner needle 16 over the entire circumference, in at least a part of an inner circumferential surface. In the close contact portion 70, both the catheter distal portion 66 and the catheter intermediate portion 68 are in close contact with the inner needle 16. Specifically, an inner circumferential surface 66a of the catheter distal portion 66 is in close contact (fits) with the outer circumferential surface of the inner needle 16 in a liquid-tight manner over the entire circumference. In addition, an inner circumferential surface 68a of the catheter intermediate portion 68 is in close contact (fits) with the outer circumferential surface of the inner needle 16 in a liquid-tight manner over the entire circumference.

A distal portion of the catheter intermediate portion 68 has a smaller outer diameter than a proximal portion of the catheter intermediate portion 68. The outer diameter of the proximal portion of the catheter intermediate portion 68 is larger than the maximum outer diameter of the catheter distal portion 66. The proximal portion of the catheter intermediate portion 68 is separated from the outer circumferential surface of the inner needle 16 over the entire circumference. A distal-most portion of the catheter intermediate portion 68 is located on the proximal side of a distal-most portion of the catheter distal portion 66. The proximal end 44a of the introduction path 44 is provided on the proximal side of a proximal-most portion of a portion (portion forming the close contact portion 70) in close contact with the outer circumferential surface of the inner needle 16 in the catheter intermediate portion 68.

In this manner, both the catheter distal portion 66 and the catheter intermediate portion 68 are in close contact with the inner needle 16 in the close contact portion 70. With this configuration, an appropriate fitting force between the inner needle 16 and the catheter 12c can be obtained.

Incidentally, the same or similar functions and effects as those of the first embodiment or the second embodiment can be obtained in the third embodiment for common parts with the first embodiment or the second embodiment.

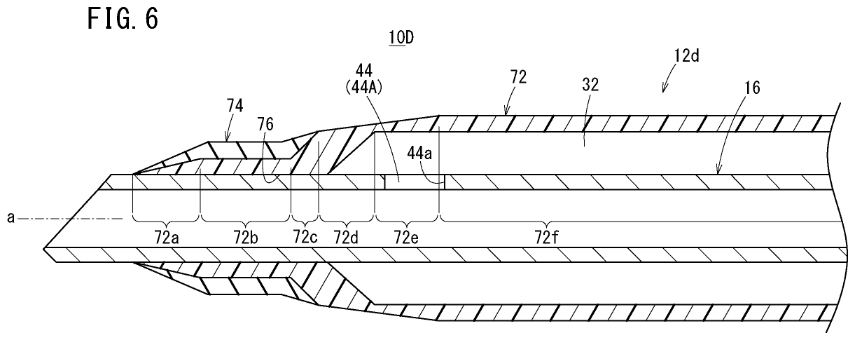

In a catheter assembly 10D according to a fourth embodiment illustrated in FIG. 6, a catheter 12d includes a plurality of sites that can be distinguished from each other along the axial direction. The plurality of sites have different hardness (elastic moduli) between adjacent sites. Specifically, the catheter 12d includes a catheter body 72 and a flexible portion 74 provided at a distal portion of the catheter body 72 as the plurality of sites.

The catheter body 72 includes: a first main body portion 72a including a distal-most portion of the catheter 12d; a second main body portion 72b adjacent to the proximal side of the first main body portion 72a; a third main body portion 72c adjacent to the proximal side of the second main body portion 72b; a fourth main body portion 72d adjacent to the proximal side of the third main body portion 72c; a fifth main body portion 72e adjacent to the proximal side of the fourth main body portion 72d; and a sixth main body portion 72f adjacent to the proximal side of the fifth main body portion 72e.

The first main body portion 72a has a tapered shape of which outer diameter decreases in the distal direction. The second main body portion 72b has a straight shape of which outer diameter is constant along the axial direction. A radial thickness of the second main body portion 72b (a wall thickness of a circumferential wall portion forming the second main body portion 72b) is thinner than the maximum radial thickness of the flexible portion 74. The third main body portion 72c has a tapered shape of which outer diameter decreases in the distal direction. The fourth main body portion 72d has a tapered shape of which outer diameter and inner diameter decrease in the distal direction. The fifth main body portion 72e has a shape having an outer diameter decreasing in the distal direction and a constant inner diameter. The sixth main body portion 72f has a straight shape of which outer diameter and inner diameter are constant along the axial direction.

The flexible portion 74 includes the distal-most portion of the catheter 12d and is configured to be more flexible than the catheter body 72. The maximum outer diameter of the flexible portion 74 is smaller than the maximum outer diameter of the catheter body 72 (the outer diameter of the sixth main body portion 72f). The catheter body 72 is interposed between the flexible portion 74 and the inner needle 16 over the entire length of the flexible portion 74. Therefore, an inner circumferential surface of the flexible portion 74 is not in close contact with an outer circumferential surface of the inner needle 16.

The catheter 12d has a close contact portion 76 that is in close contact with the outer circumferential surface of the inner needle 16 over the entire circumference. In the close contact portion 76, only the inner circumferential surface of the catheter body 72 between the catheter body 72 and the flexible portion 74 is in close contact with the outer circumferential surface of the inner needle 16.

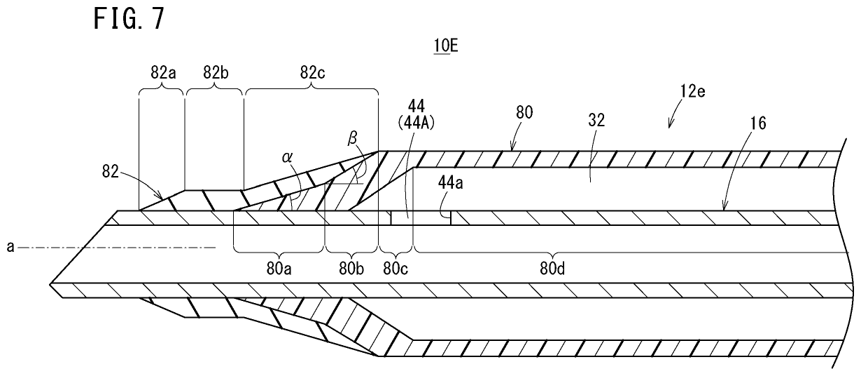

In a catheter assembly 10E according to a fifth embodiment illustrated in FIG. 7, a catheter 12e includes a plurality of sites that can be distinguished from each other along the axial direction. The plurality of sites have different hardness (elastic moduli) between adjacent sites. Specifically, the catheter 12e includes a catheter body 80 and a flexible portion 82 provided at a distal portion of the catheter body 80 as the plurality of sites.

The catheter body 80 includes: a first main body portion 80a including a distal-most portion of the catheter body 80; a second main body portion 80b adjacent to the proximal side of the first main body portion 80a; a third main body portion 80c adjacent to the proximal side of the second main body portion 80b; and a fourth main body portion 80d adjacent to the proximal side of the third main body portion 80c.

The first main body portion 80a has a tapered shape of which outer diameter decreases in the distal direction. The second main body portion 80b has a tapered shape of which outer diameter decreases in the distal direction. The third main body portion 80c has a shape in which an outer diameter is constant along the axial direction and an inner diameter decreases in the distal direction. The fourth main body portion 80d has a straight shape of which outer diameter and inner diameter are constant along the axial direction.

An inclination angle α of an outer circumferential surface of the first main body portion 80a with respect to the axis a of the catheter 12e is smaller than an inclination angle β of an outer circumferential surface of the second main body portion 80b with respect to the axis a of the catheter 12e. In another aspect, the inclination angle α of the outer circumferential surface of the first main body portion 80a with respect to the axis a of the catheter 12e may be the same as the inclination angle β of the outer circumferential surface of the second main body portion 80b with respect to the axis a of the catheter 12e.

The flexible portion 82 is configured to be more flexible than the catheter body 80. The flexible portion 82 includes: a first flexible portion 82a including a distal-most portion of the flexible portion 82; a second flexible portion 82b adjacent to the proximal side of the first flexible portion 82a; and a third flexible portion 82c adjacent to the proximal side of the second flexible portion 82b. The first flexible portion 82a has a tapered shape of which outer diameter decreases in the distal direction. The second flexible portion 82b has a straight shape of which outer diameter is constant in the axial direction. The third flexible portion 82c has a tapered shape of which outer diameter decreases in the distal direction.

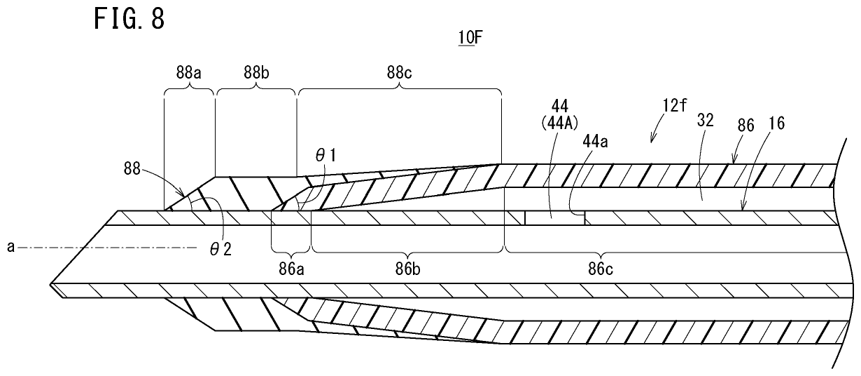

In a catheter assembly 10F according to a sixth embodiment illustrated in FIG. 8, a catheter 12f includes a plurality of sites that can be distinguished from each other along the axial direction. The plurality of sites have different hardness (elastic moduli) between adjacent sites. Specifically, the catheter 12f includes a catheter body 86 and a flexible portion 88 provided at a distal portion of the catheter body 86 as the plurality of sites.

The catheter body 86 includes: a first main body portion 86a including a distal-most portion of the catheter body 86; a second main body portion 86b adjacent to the proximal side of the first main body portion 86a; and a third main body portion 86c adjacent to the proximal side of the second main body portion 86b.

The first main body portion 86a has a tapered shape of which outer diameter decreases in the distal direction. The second main body portion 86b has a tapered shape of which outer diameter and inner diameter decrease in the distal direction. The third main body portion 86c has a straight shape of which outer diameter and inner diameter are constant along the axial direction.

The flexible portion 88 is configured to be more flexible than the catheter body 86. The flexible portion 88 includes: a first flexible portion 88a including a distal-most portion of the flexible portion 88; a second flexible portion 88b adjacent to the proximal side of the first flexible portion 88a; and a third flexible portion 88c adjacent to the proximal side of the second flexible portion 88b. The first flexible portion 88a has a tapered shape of which outer diameter decreases in the distal direction. The second flexible portion 88b has a straight shape of which outer diameter is constant along the axial direction. The third flexible portion 88c has a tapered shape of which outer diameter and inner diameter decrease in the distal direction.

An inclination angle θ1 of an outer circumferential surface of the first main body portion 86a with respect to the axis a of the catheter 12f is the same as an inclination angle θ2 of an outer circumferential surface of the first flexible portion 88a with respect to the axis a of the catheter 12f. In another aspect, the inclination angle θ1 of the outer circumferential surface of the first main body portion 86a with respect to the axis a of the catheter 12f is larger than the inclination angle θ2 of the outer circumferential surface of the first flexible portion 88a with respect to the axis a of the catheter 12f.

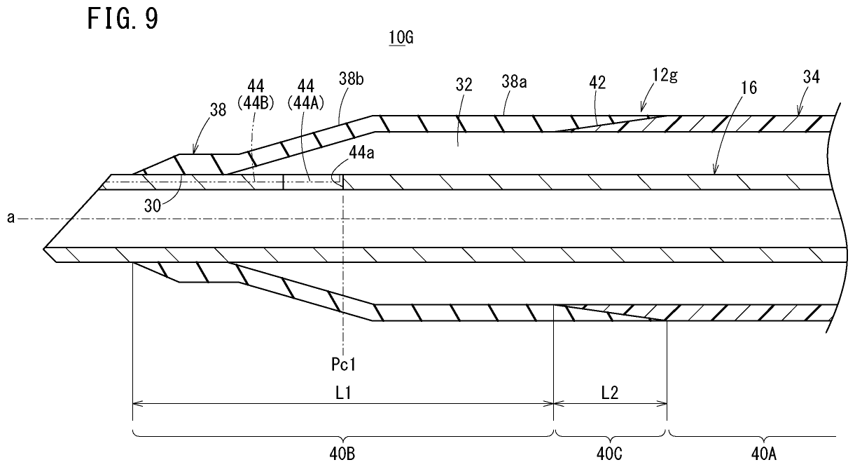

In a catheter assembly 10G according to a seventh embodiment illustrated in FIG. 9, at least the proximal end 44a of the introduction path 44 in the catheter 12g in the initial state is provided on the distal side of the distal-most portion of the single catheter body region 40A. The entire side hole 44A illustrated in FIG. 9 is provided on the distal side of the distal-most portion of the single catheter body region 40A. More specifically, the proximal end 44a of the introduction path 44 is provided at the same position in the axial direction as the axial center position Pc1 of the single flexible portion region 40B. The proximal end 44a of the introduction path 44 is provided on the distal side of a proximal end of the tapered portion 38b of the flexible portion 38. The side hole 44A is provided at a position where the entire side hole 44A opposes an inner circumferential surface of the tapered portion 38b of the flexible portion 38. In another aspect, the proximal end 44a (or the entire side hole 44A) of the introduction path 44 may be provided at a position opposing an inner circumferential surface of the straight portion 38a of the flexible portion 38.

The present invention is not limited to the above-described embodiment, and various modifications can be made within a scope not departing from a gist of the present invention.

What is claimed is:

1. A catheter assembly comprising:
a catheter; and
an inner needle inserted through the catheter, wherein:
the catheter comprises a plurality of sites that are distinguishable from each other along an axial direction, and
the plurality of sites have different hardnesses between adjacent sites.

2. The catheter assembly according to claim 1, wherein:
the catheter comprises a catheter body and a flexible portion that includes a distal-most portion of the catheter body, as the plurality of sites, and
the flexible portion is more flexible than the catheter body.

3. The catheter assembly according to claim 2, wherein:
a flow path for flashback confirmation is formed between the catheter and the inner needle,
the inner needle comprises an introduction path that communicates with the flow path to introduce blood into the flow path, and
a proximal end of the introduction path is located on a distal side of an axial center position of a portion of the flexible portion present on a distal side of a distal-most portion of the catheter body.

4. The catheter assembly according to claim 2, wherein:
a flow path for flashback confirmation is formed between the catheter and the inner needle,
the inner needle comprises an introduction path that communicates with the flow path to introduce blood into the flow path, and
a proximal end of the introduction path is located at a similar position in the axial direction as an axial center position of a portion of the flexible portion present on a distal side of a distal-most portion of the catheter body.

5. The catheter assembly according to claim 1, wherein:
the catheter comprises a catheter body, a catheter distal portion that includes a distal-most portion of the catheter, and a catheter intermediate portion located between the catheter body and the catheter distal portion, as the plurality of sites.

6. The catheter assembly according to claim 5, wherein:
a flow path for flashback confirmation is formed between the catheter and the inner needle,
the inner needle comprises an introduction path that communicates with the flow path to introduce blood into the flow path, and
a proximal end of the introduction path is located on a distal side of an axial center position of a portion, present on a distal side of a distal-most portion of the catheter body, in a combined region of the catheter distal portion and the catheter intermediate portion.

7. The catheter assembly according to claim 5, wherein:
the catheter comprises a close contact portion, which is in close contact with an outer circumferential surface of the inner needle, in at least a part of an inner circumferential surface, and
both the catheter distal portion and the catheter intermediate portion are in close contact with the inner needle at the close contact portion.

8. A catheter assembly comprising:
a catheter; and
an inner needle inserted through the catheter,
wherein the catheter comprises a catheter body and a flexible portion located at a distal portion of the catheter body,
wherein the flexible portion includes a distal-most portion of the catheter and is more flexible than the catheter body.

9. The catheter assembly according to claim 8, wherein:
a flow path for flashback confirmation is formed between the catheter and the inner needle,
the inner needle comprises an introduction path that communicates with the flow path to introduce blood into the flow path, and
a proximal end of the introduction path is located on a distal side of an axial center position of a portion of the flexible portion present on a distal side of a distal-most portion of the catheter body.

10. The catheter assembly according to claim 8, wherein:
a flow path for flashback confirmation is formed between the catheter and the inner needle,
the inner needle comprises an introduction path that communicates with the flow path to introduce blood into the flow path, and
a proximal end of the introduction path is located at a similar position in the axial direction as an axial center position of a portion of the flexible portion present on a distal side of a distal-most portion of the catheter body.

11. A catheter assembly comprising:
a catheter; and
an inner needle inserted through the catheter,
wherein the catheter comprises a catheter body, a catheter distal portion that includes a distal-most portion of the catheter, and a catheter intermediate portion located between the catheter body and the catheter distal portion, and
wherein the catheter distal portion is more flexible than the catheter body.

12. The catheter assembly according to claim 11, wherein:
a flow path for flashback confirmation is formed between the catheter and the inner needle,
the inner needle comprises an introduction path that communicates with the flow path to introduce blood into the flow path, and
a proximal end of the introduction path is located on a distal side of an axial center position of a portion, present on a distal side of a distal-most portion of the catheter body, in a combined region of the catheter distal portion and the catheter intermediate portion.

13. The catheter assembly according to claim 11, wherein:
the catheter comprises a close contact portion, which is in close contact with an outer circumferential surface of the inner needle, in at least a part of an inner circumferential surface, and
both the catheter distal portion and the catheter intermediate portion are in close contact with the inner needle at the close contact portion.
